(19) United States
(12) Patent Application Publication
(10) Pub. No.: US 2021/0391085 A1
(43) Pub. Date: Dec. 16, 2021

(54) MACHINE LEARNING TECHNIQUES FOR GENERATING ICU PREDICTIONS

(71) Applicant: EQ IP Holdings LLC, Wilmington, DE (US)

(72) Inventor: Mario V. Fusaro, New York, NY (US)

(21) Appl. No.: 17/349,244

(22) Filed: Jun. 16, 2021

Related U.S. Application Data
(60) Provisional application No. 63/039,604, filed on Jun. 16, 2020.

(51) Int. Cl. G16H 50/30; G16H 50/20; A61B 5/0205

(52) U.S. Cl. CPC G16H 50/30; A61B 2505/03; A61B 5/02055; G16H 50/20

(57) ABSTRACT

A computing device may receive a first one or more features associated with a patient. The computing device may determine, based on the first one or more features, a second one or more features associated with the patient. The determining the second one or more features associated with the patient may comprise feature engineering to generate features that improve the accuracy of one or more machine learning models. For example, the second one or more features may be determined based on a combination of at least a subset of the first one or more features. The computing device may generate, based on the one or more machine learning models, a prediction associated with the patient, which may comprise a mortality rate for the patient in the intensive care unit (ICU) or a length of stay in the ICU for the patient.

### CROSS REFERENCE TO RELATED APPLICATIONS

This application claims the benefit of U.S. Provisional Patent Application No. 63/039,604, filed Jun. 16, 2020, the contents of which are incorporated by reference in their entirety as if fully set forth herein.

### TECHNICAL FIELD

The present invention relates to machine learning techniques and in particular improved decision tree models.

### BACKGROUND

Many hospitals include an intensive care unit (ICU). ICUs handle patients with severe or life-threatening illnesses. Data associated with an ICU, such as the mortality rates of patients and lengths of stay (LOS), is often used to compare ICUs' performance. However, these comparisons are often of questionable value because each hospital is treating a different set of patients. For example, one hospital may be treating a large number of very sick patients while another hospital may be treating a much lower number of sick patients. In this example, ICU comparisons of these hospitals, based on the data associated with the patients of each respective ICU, would be of little value. Accordingly, there is a need for improved techniques for generating data associated with an ICU in order to evaluate ICU performance and improve patient care.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

In accordance with one embodiment, a computing device configured with one or more machine learning models may receive a first one or more features associated with a patient. The first one or more features associated with the patient may comprise one or more of types of data associated with the patient. The computing device may determine, based on the first one or more features, a second one or more features associated with the patient. The determining the second one or more features associated with the patient may comprise feature engineering to generate features that improve the accuracy of the one or more machine learning models. For example, the second one or more features may be determined based on a combination of at least a subset of the first one or more features. The computing device may generate, based on the one or more machine learning models, a prediction associated with the patient. The one or more machine learning models may comprise at least one or a classifier and a regressor trained and tested based on the first one or more features and the second one or more features. The prediction may comprise a mortality rate for the patient in the ICU or a length of stay in the ICU for the patient.

### BRIEF DESCRIPTION OF THE DRAWINGS

In order to facilitate a more robust understanding of the application, reference is now made to the accompanying drawings, in which like elements are referenced with like numerals. These drawings should not be construed to limit the application and are intended only to be illustrative.

FIG. 1 is a diagram of an example of method;

FIG. 2 is a diagram of an example system that can implement the machine learning techniques described herein in accordance with one embodiment.

### DETAILED DESCRIPTION OF ILLUSTRATIVE EMBODIMENTS

Described herein are machine learning models configured to predict a mortality rate and predict a length of stay for a patient in an ICU. The machine learning models may be based on one or more of linear regression algorithms, logistic regression algorithms, or decision tree algorithms. The machine learning models may be generated using training data and testing data. The training data and testing data comprises patient population data available in an open source database.

The patient population for training and testing may be from the MIMIC cohort in the Beth Israel Deaconess Hospital. This patient population data is available in an open source database (“MIMIC database”), which may be used in training and testing the machine learning models described herein. In this set of patient population data, the patient population was aged 16 to 89 years of age and spent a minimum of twelve hours in an ICU. The population was placed in a Medical Intensive Care Unit (MICU), Surgical ICU (SICU), Cardiac Surgery Resuscitation Unit (CSRU), Cardiac Care Unit (CCU), Trauma SICU (SICU) over 2001-2008.

The machine learning models may be trained based on a plurality of features extracted from the patient population data of the MIMIC database. The plurality of features may be extracted using one or more queries. The queries may be based on SQL code. For example, the plurality of features may be extracted using 70 queries generated using SQL code. The queries may be enabled by technology such as Google BigQuery.

The extracted plurality of features comprises a maximum, minimum, average or presence/absence over the first 30 hours of ICU admission. Demographic information and length of stay (LOS) data were captured from time of admission. The initial 74 features (independent variables) used in generating the machine learning models describe herein comprise: ‘First Care Unit’, ‘Transferring Service at ICU admission’, ‘Pre ICU LOS’, ‘ICU LOS’, ‘Hospital LOS’, ‘Hospital Mortality’, ‘ICU Mortality’, ‘Age’, ‘Gender’, ‘Emergency/Urgent Admission’, ‘Intubation’, ‘Number of previous ICU admits’, ‘Average Positive End Expiratory Pressure (PEEP), ‘Maximum PEEP’, ‘Driving Pressure’, ‘Inspiratory Positive Airway Pressure (IPAP), ‘Mean Arterial Pressure (MAP)’, ‘Average Systolic Blood Pressure (BP)’, ‘Average Diastolic BP’, ‘Average Heart Rate’, ‘Average Respiratory Rate’, ‘Average Temperature (F)’, ‘Minimum Temperature (F)’, ‘Maximum Temperature (F)’, ‘Average pH’, ‘Minimum pH’, ‘Average Lactate’, ‘Maximum Lactate’, ‘Average Albumin’, ‘Average Anion Gap (AG)’, ‘Maximum AG’, ‘Average Creatinine’, ‘Maximum Creatinine’, ‘Average White Blood Cell (Avg WBC)’, ‘Minimum WBC’, ‘Average Prothrombin Time (PT)’, ‘Average Hemoglobin (Avg Hgb)’, ‘Minimum Hemoglobin’, ‘Average Platelet Count (Avg Plt)’, ‘Average PaO2’, ‘Average FiO2’, ‘Average Sodium’, ‘Average Total Bilirubin’, ‘Average Serum Glucose’, ‘Maximum Serum Glucose’, ‘Minimum Serum Glucose’, ‘Average Serum Potassium’, ‘Average Troponin T/I’, ‘Maximum Troponin T/I’, ‘Average Bands’, ‘Maximum Bands’, ‘Blood Culture+’, ‘Average Glasgow Coma Score’, ‘Chemotherapy Agents’, ‘Inotropes’, ‘Pressors’, ‘Acetylcysteine’, ‘Bicarbonate Drip’, ‘Tracheostomy’, ‘Antibiotics’, ‘Respiratory Therapies’, ‘Invasive Cardiac Procedures’, ‘Neurologic Agents’, ‘Blood Pressure Drips’, ‘Renal Replacement Therapy’, ‘Hematologic Agents’, ‘Average Blood Urea Nitrogen (BUN)’, ‘Maximum BUN’, ‘P/F’, ‘MELD Score’, ‘HRMAP’, ‘EmergETT’, ‘DIC’, ‘Pancytopenia’.

A subset of features of the extracted plurality of features may be engineered to improve machine learning model performance. Some features of the extracted plurality of features on their own may not be meaningful with respect to indicating a disease or condition relevant to mortality rate in an ICU or length of stay in an ICU. Accordingly, these features may not contribute to predictions generated by the machine learning models. However, these features may be engineered (e.g., combined and/or otherwise transformed) with other features into one feature that is indicative of a disease or condition relevant to mortality rate of length of stay. The engineered features may then be used in the machine learning generation process described herein and result in improved performance of the machine learning models described herein.

The engineering of a subset of features of the extracted plurality of features may comprise transforming each feature in the subset from their original format (e.g., categorical (e.g., yes/no) or numerical/continuous data) into a single number. That generated single number may then be combined with one or more other features of the extracted plurality of features.

The engineered features used in generating the machine learning models describe herein comprise: ‘Inotropes’, ‘Pressors’, ‘Invasive Cardiac Procedures’, ‘Neurologic Agents’, ‘Blood Pressure Drips’, ‘MELD Score’, ‘P/F’, ‘HRMAP’, ‘EmergETT’, ‘DIC’, ‘Pancytopenia’.

Inotropes' may be scored as an ordinal input where dopamine and dobutamine use is one point each. ‘Pressors’ may denote use of phenylephrine, epinephrine, or norepinephrine and are worth one point each except for norepinephrine, which is worth four points determined by logistic regression coefficients with regard to these three features and mortality. ‘Invasive Cardiac Procedures’ ordinal points may be determined as above and include intraaortic balloon pump worth one point, impella and cardiobypass worth two points, left ventricular assist device worth three points, and extracorporeal membrane oxygenation worth eight points. ‘Neurologic Agents’ may include mannitol use worth six points, hypertonic saline worth one point, electroencephalogram use worth three points, intracranial pressure monitoring worth four points. ‘Blood Pressure Drips’ include nitroprusside, nicardipine, fenoldopam, nitroglycerine all worth one point. ‘MELD’ score may be calculated as follows:

[10*((0.957+LN(Avg Cr))+(0.378*LN(Tot Bili))+(1.12*LN(PT/13)))+6.43]

‘P/F’ may be calculated as PaO2/FiO2. ‘HRMAP’ may simulate an acute shock state calculated as HR/MAP. ‘EmergETT’ may incorporate both emergent admission and intubation on an ordinal scale from 0 to 2 where nonelective admissions may be worth one point and intubation in first 30 hours is worth one point. ‘DIC’ may aim to simulate disseminated intravascular coagulation and may be calculated as [PT/(Avg Plt)/15]. Lastly, ‘Pancytopenia’ is calculated as [Avg Hgb+Avg WBC+(Avg Plt/15)].

Based on the extracted plurality of features and the engineered features, the machine learning models may be generated. A first machine learning model may comprise a classifier configured to predict a mortality rate for a patient in a hospital and/or ICU of the hospital. The machine learning classifier may predict a number between 0 and 1 indicating a mortality rate. The machine learning classifier may be based on a decision tree algorithm. The decision tree algorithm may be based on XGBoost. The machine learning classifier may be referred to herein to as an XGB tree classifier.

The XGBoost tree classifier may determine a learning rule discriminating both ICU mortality and hospital mortality. For example, a Python script may be written and hyperparameter tuning may be performed as follows for the machine learning classifier:
(max_depth=4, learning_rate=0.1, n_estimators=205, objective=‘binary:logistic’, booster=‘gbtree’, n_jobs=1, nthread=None, gamma=0, min_child_weight=1, max_delta_step=0, sub_sample=1, col_sample_bytree=1, colsample_bylevel=1, col_sample_bynode=1, reg_alpha=0, reg_lambda=1, scale_pos_weight=1, base score=0.5, random_state=0, seed=None, missing=None)

Following training and testing of the machine learning classifier and assessment of the accuracy of its predictions of ICU and hospital mortality rates, 30 features of the extracted plurality of features and the engineered features were determined to be best at predicting mortality rates. During this process, features that were duplicative, highly correlated, or not useful in predicting mortality rates were removed from the extracted plurality of features. The machine learning classifier was then retrained using these 30 features to improve the accuracy of the machine learning classifier.

These 30 features determined to be best at predicting ICU and hospital mortality rates comprise: ‘Transferring Service at ICU admission’, ‘Pre ICU LOS’, ‘Age’, ‘Average PEEP, ‘Average Systolic BP’, ‘Average Diastolic BP’, ‘Average Heart Rate’, ‘Average Respiratory Rate’, ‘Average Temperature (F)’, ‘Average pH’, ‘Average Lactate’, ‘Average Albumin’, ‘Average Anion Gap (AG)’, ‘Minimum WBC’, ‘Average Hemoglobin’, ‘Average Platelet Count’, ‘Average Sodium’, ‘Average Total Bilirubin’, ‘Average BUN’, ‘Average Glasgow Coma Score’, ‘Inotropes’, ‘Pressors’, ‘Invasive Cardiac Procedures’, ‘Neurologic Agents’, ‘Blood Pressure Drips’, ‘MELD Score’, ‘HRMAP’, ‘EmergETT’, ‘DIC’, ‘Pancytopenia’.

Based on the extracted plurality of features and the engineered features, a second machine learning model may be generated. The second machine learning model may comprise a regressor (e.g., based on a linear regression algorithm) configured to predict a length of stay for a patient in a hospital and/or ICU of the hospital. The regressor may predict a continuous variable such as a number representing a predicted length of stay. The machine learning regressor may be referred to herein to as an XGB regressor.

The XGB regressor may determine a learning rule discriminating both ICU mortality and hospital length of stay. For example, a Python script may be written and hyperparameter tuning may be performed. Hyperparameters for the XGB regressor differed as follows from the XGBoost tree classifier:
[max_depth=6, learning_rate=0.1, n_estimators=78, verbosity=1, silent=None, objective=‘reg:squarederror’, booster=‘gbtree’]

Following training and testing of the machine learning regressor and assessment of the accuracy of its predictions of ICU and hospital lengths of stay, 36 features of the extracted plurality of features and the engineered features were determined to be best at predicting lengths of stay. During this process, features that were duplicative, highly correlated, or not useful in predicting lengths of stay were removed from the extracted plurality of features. The machine learning regressor was then retrained using these 36 features to improve the accuracy of the machine learning regressor.

These 36 features determined to be best at predicting ICU and hospital length of stay comprise: ‘Transferring Service at ICU admission’, ‘Pre ICU LOS’, ‘Age’, ‘Emergency/Urgent Admission’, ‘Average PEEP’, ‘Driving Pressure’, ‘IPAP’, ‘FiO2’, ‘Average Systolic BP’, ‘Average Diastolic BP’, ‘Average Heart Rate’, ‘Average Respiratory Rate’, ‘Average Temperature (F)’, ‘Average pH’, ‘Average Lactate’, ‘Average Albumin’, ‘Average Anion Gap (AG)’, ‘Average Creatinine (Avg Cr)’, ‘Minimum WBC’, ‘Average Hemoglobin’, ‘Average Platelet Count’, ‘Average Sodium’, ‘Average Total Bilirubin (Tot Bili)’, ‘Average BUN’, ‘Average Glasgow Coma Score’, ‘Inotropes’, ‘Pressors’, ‘Invasive Cardiac Procedures’, ‘Neurologic Agents’, ‘Blood Pressure Drips’, ‘MELD Score’, ‘P/F’, ‘HRMAP’, ‘EmergETT’, ‘DIC’, ‘Pancytopenia’.

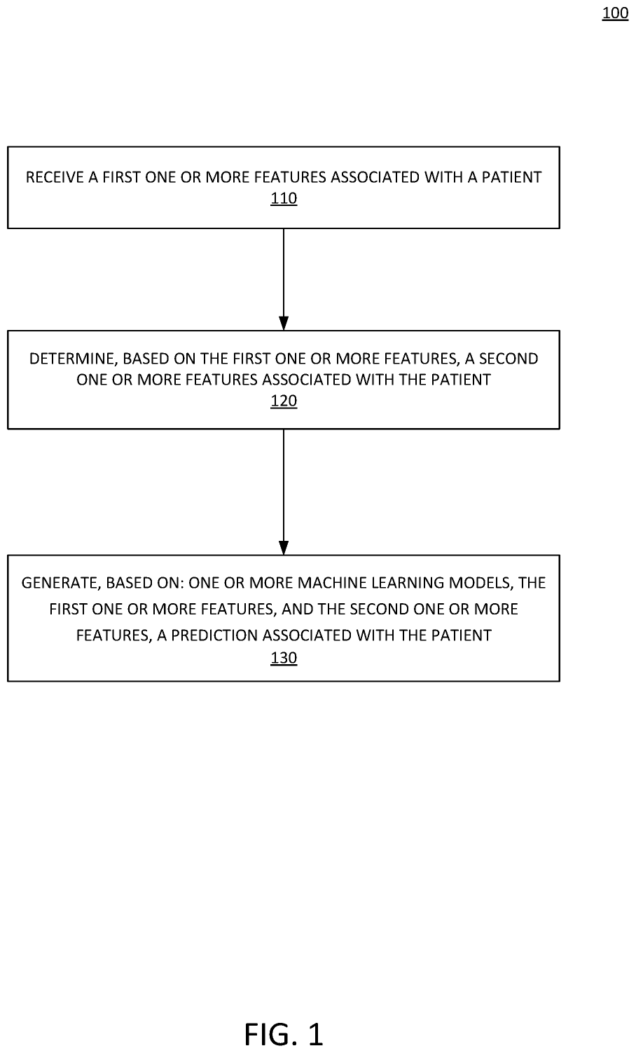

FIG. 1 is a diagram of an example method 100. A computing device configured with one or more machine learning models described above may receive a first one or more features associated with a patient (step 110). The first one or more features associated with the patient may comprise one or more of: Transferring Service at ICU admission, Pre ICU length of stay, Age, Average PEEP, Average Systolic BP, Average Diastolic BP, Average Heart Rate, Average Respiratory Rate, Average Temperature (F), Average pH, Average Lactate, Average Albumin, Average Anion Gap (AG), Minimum WBC, Average Hemoglobin, Average Platelet Count, Average Sodium, Average Total Bilirubin, Average BUN, or Average Glasgow Coma Score. Alternatively or additionally, the first one or more features associated with the patient may comprise one or more of: Transferring Service at ICU admission, Pre ICU length of stay, Age, Emergency/Urgent Admission, Average PEEP, Driving Pressure, IPAP, FiO2, Average Systolic BP, Average Diastolic BP, Average Heart Rate, Average Respiratory Rate, Average Temperature (F), Average pH, Average Lactate, Average Albumin, Average Anion Gap (AG), Average Creatinine (Avg Cr), Minimum WBC, Average Hemoglobin, Average Platelet Count, Average Sodium, Average Total Bilirubin (Tot Bili), Average BUN, or Average Glasgow Coma Score.

The computing device may determine, based on the first one or more features, a second one or more features associated with the patient (step 120). The determining the second one or more features associated with the patient may comprise feature engineering as described above. For example, the second one or more features may be determined based on a combination of at least a subset of the first one or more features. The second one or more features may comprise one or more of: Inotropes, Pressors, Invasive Cardiac Procedures, Neurologic Agents, Blood Pressure Drips, MELD Score, P/F, HRMAP, EmergETT, DIC, or Pancytopenia.

The computing device may generate, based on one or more machine learning models, the first one or more features, and the second one or more features, a prediction associated with the patient (step 130). The one or more machine learning models may comprise at least one or a classifier and a regressor trained and tested as described above. The prediction may comprise a mortality rate for the patient in the ICU or a length of stay in the ICU for the patient.

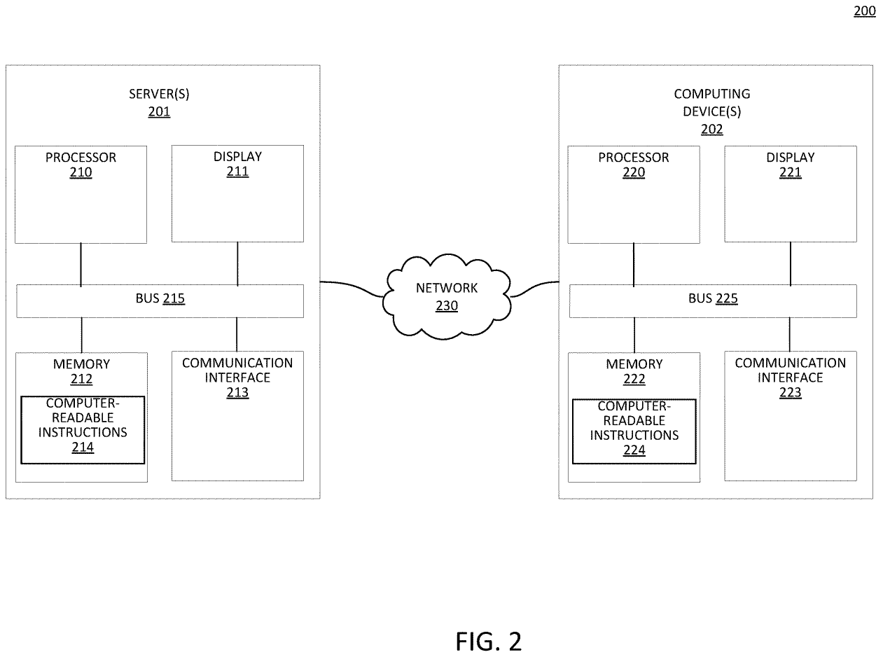

FIG. 2 is a diagram of an example system 200 that can implement the machine learning models described herein in accordance with one embodiment. The example system 200 can comprise server(s) 201, which can comprise processor 210, memory 212, display 211, and communication interface 213, which can be in communication with one another and with any other associated components of server(s) 201 using any suitable type of communication such as, for example, via bus 215. Communication interface 213 can comprise a receiver, transmitter, or transceiver that is capable of communicating in a wired or wireless network 230. Memory 212 can be any computer-readable medium or suitable device for electronic data storage including but not limited to a database and computer-readable instructions 214. Processor 210 can be configured to execute computer-readable instructions 214 causing server(s) 201 to perform the procedures for predicting ICU mortality rate and length of stay in accordance with the embodiments described herein. Server(s) 201 can host websites accessible to provide the patient data described herein.

The example system 200 can comprise computing device(s) 202, which can comprise processor 220, memory 222, display 221, and communication interface 223, which can be in communication with one another and with any other associated components of computing device(s) 202 using any suitable type of communication such as, for example, via bus 225. Communication interface 223 can comprise a receiver, transmitter, or transceiver that is capable of communicating in a wired or wireless network 230. Memory 222 can be any computer-readable medium or suitable device for electronic data storage. Processor 220 can be configured to execute computer-readable instructions 224 causing computing device(s) 202 to perform the procedures for predicting ICU mortality rate and length of stay in accordance with the embodiments described herein. Computer-readable instructions 224 can be stored in memory 222. By way of non-limiting example, computing device(s) 202 can include one or more of a desktop computer, a laptop computer, a handheld computer, a tablet, a netbook, a smartphone, a gaming console, and/or other computing platforms.

Having thus described the various embodiments, it is to be appreciated and will be apparent to those skilled in the art that the present embodiments are to be considered in all respects as illustrative and not restrictive. Although features and elements are described above in particular combinations, it is to be appreciated that each feature or element can be used alone or in any combination or sub-combination with or without the other features and elements. Any single embodiment described herein can be supplemented with one or more elements from any one or more of the other embodiments described herein. Any single element of an embodiment can be replaced with one or more elements from any one or more of the other embodiments described herein.

What is claimed:

1. A method comprising:
receiving a first one or more features associated with a patient;
determining, based on the first one or more features, a second one or more features associated with the patient; and
generating, based on:
one or more machine learning models,
the first one or more features, and
the second one or more features,
a prediction associated with the patient.

2. The method of claim 1, wherein the prediction indicates a mortality rate for the patient in an intensive care unit (ICU).

3. The method of claim 1, wherein the prediction indicates a length of stay in an intensive care unit (ICU) for the patient.

4. The method of claim 1, wherein the first one or more features comprise one or more of: Transferring Service at ICU admission, Pre ICU length of stay, Age, Average PEEP, Average Systolic BP, Average Diastolic BP, Average Heart Rate, Average Respiratory Rate, Average Temperature (F), Average pH, Average Lactate, Average Albumin, Average Anion Gap (AG), Minimum WBC, Average Hemoglobin, Average Platelet Count, Average Sodium, Average Total Bilirubin, Average BUN, or Average Glasgow Coma Score.

5. The method of claim 1, wherein the first one or more features comprise one or more of: Transferring Service at ICU admission, Pre ICU length of stay, Age, Emergency/Urgent Admission, Average PEEP, Driving Pressure, IPAP, FiO2, Average Systolic BP, Average Diastolic BP, Average Heart Rate, Average Respiratory Rate, Average Temperature (F), Average pH, Average Lactate, Average Albumin, Average Anion Gap (AG), Average Creatinine (Avg Cr), Minimum WBC, Average Hemoglobin, Average Platelet Count, Average Sodium, Average Total Bilirubin (Tot Bili), Average BUN, or Average Glasgow Coma Score.

6. The method of claim 1, wherein the second one or more features comprise one or more of: Inotropes, Pressors, Invasive Cardiac Procedures, Neurologic Agents, Blood Pressure Drips, MELD Score, P/F, HRMAP, EmergETT, DIC, or Pancytopenia.

7. The method of claim 1, wherein the second one or more features may be determined based on a combination of at least a subset of the first one or more features.

8. A non-transitory computer-readable medium having instructions stored therein which, when executed by a computer, cause the computer to perform operations comprising:
receiving a first one or more features associated with a patient;
determining, based on the first one or more features, a second one or more features associated with the patient; and
generating, based on:
one or more machine learning models,
the first one or more features, and
the second one or more features,
a prediction associated with the patient.

9. The non-transitory computer-readable medium of claim 8, wherein the prediction indicates a mortality rate for the patient in an intensive care unit (ICU).

10. The non-transitory computer-readable medium of claim 8, wherein the prediction indicates a length of stay in an intensive care unit (ICU) for the patient.

11. The non-transitory computer-readable medium of claim 8, wherein the first one or more features comprise one or more of: Transferring Service at ICU admission, Pre ICU length of stay, Age, Average PEEP, Average Systolic BP, Average Diastolic BP, Average Heart Rate, Average Respiratory Rate, Average Temperature (F), Average pH, Average Lactate, Average Albumin, Average Anion Gap (AG), Minimum WBC, Average Hemoglobin, Average Platelet Count, Average Sodium, Average Total Bilirubin, Average BUN, or Average Glasgow Coma Score.

12. The non-transitory computer-readable medium of claim 8, wherein the first one or more features comprise one or more of: Transferring Service at ICU admission, Pre ICU length of stay, Age, Emergency/Urgent Admission, Average PEEP, Driving Pressure, IPAP, FiO2, Average Systolic BP, Average Diastolic BP, Average Heart Rate, Average Respiratory Rate, Average Temperature (F), Average pH, Average Lactate, Average Albumin, Average Anion Gap (AG), Average Creatinine (Avg Cr), Minimum WBC, Average Hemoglobin, Average Platelet Count, Average Sodium, Average Total Bilirubin (Tot Bili), Average BUN, or Average Glasgow Coma Score.

13. The non-transitory computer-readable medium of claim 8, wherein the second one or more features comprise one or more of: Inotropes, Pressors, Invasive Cardiac Procedures, Neurologic Agents, Blood Pressure Drips, MELD Score, P/F, HRMAP, EmergETT, DIC, or Pancytopenia.

14. The non-transitory computer-readable medium of claim 8, wherein the second one or more features may be determined based on a combination of at least a subset of the first one or more features.

15. A computing device comprising:
one or more processors; and
memory storing instructions that, when executed by the one or more processors, cause the device to:
receive a first one or more features associated with a patient;
determine, based on the first one or more features, a second one or more features associated with the patient; and
generate, based on:
one or more machine learning models,
the first one or more features, and
the second one or more features,
a prediction associated with the patient.

16. The computing device of claim 15, wherein the prediction indicates: a mortality rate for the patient in an intensive care unit (ICU), or a length of stay in an intensive care unit (ICU) for the patient.

17. The computing device of claim 15, wherein the first one or more features comprise one or more of: Transferring Service at ICU admission, Pre ICU length of stay, Age, Average PEEP, Average Systolic BP, Average Diastolic BP, Average Heart Rate, Average Respiratory Rate, Average Temperature (F), Average pH, Average Lactate, Average Albumin, Average Anion Gap (AG), Minimum WBC, Average Hemoglobin, Average Platelet Count, Average Sodium, Average Total Bilirubin, Average BUN, or Average Glasgow Coma Score.

18. The computing device of claim 15, wherein the first one or more features comprise one or more of: Transferring Service at ICU admission, Pre ICU length of stay, Age, Emergency/Urgent Admission, Average PEEP, Driving Pressure, IPAP, FiO2, Average Systolic BP, Average Diastolic BP, Average Heart Rate, Average Respiratory Rate, Average Temperature (F), Average pH, Average Lactate, Average Albumin, Average Anion Gap (AG), Average Creatinine (Avg Cr), Minimum WBC, Average Hemoglobin, Average Platelet Count, Average Sodium, Average Total Bilirubin (Tot Bili), Average BUN, or Average Glasgow Coma Score.

19. The computing device of claim 15, wherein the second one or more features comprise one or more of: Inotropes, Pressors, Invasive Cardiac Procedures, Neurologic Agents, Blood Pressure Drips, MELD Score, P/F, HRMAP, EmergETT, DIC, or Pancytopenia.

20. The computing device of claim 15, wherein the second one or more features may be determined based on a combination of at least a subset of the first one or more features.
